(19) United States
(12) Patent Application Publication
(10) Pub. No.: US 2007/0178500 A1
(43) Pub. Date: Aug. 2, 2007

(54) METHODS OF DETERMINING RELATIVE GENETIC LIKELIHOODS OF AN INDIVIDUAL MATCHING A POPULATION

(76) Inventors: Lucas MARTIN, Arlington, VA (US); Eduardas Valaitis, Arlington, VA (US)

Correspondence Address: CASTELLANO PLLC, P.O. Box 1555, Great Falls, VA, 22066, US

(21) Appl. No.: 11/621,646

(22) Filed: Jan. 10, 2007

Related U.S. Application Data
(60) Provisional application No. 60/766,426, filed on Jan. 18, 2006.

(51) Int. Cl. C12Q 1/68; G06F 19/00

(52) U.S. Cl. 435/6; 702/20

(57) ABSTRACT

Provided are methods of determining an individual's relative likelihood of having a genetic match with one or more local populations as compared to a generic index population. Also provided are systems, apparatuses, kits, and machine-readable medium relating to such methods. The methods may be used for example, to identify an individual's or individual's ancestor's most likely geographic origin, or to identify the breed, species, kingdom, etc. of an organism.

### RELATED APPLICATION

This patent application claims the benefit of priority to U.S. Provisional Patent Application No. 60/766,426 filed on Jan. 18, 2006, entitled “GeoGenetic Profile.” The provisional patent application is hereby incorporated by reference in its entirety, including all text and figures.

### FIELD

Exemplary embodiments of the present invention are generally directed to methods of determining an individual's relative likelihood of having a genetic match with one or more local populations, as compared to a generic index population. The individual may be a human or any other organism.

Methods of the invention may be used for example to identify an individual person's most likely geographic origin or the most likely geographic origin of an individual's ancestors. Such uses may be desirable for example with respect to law enforcement or for genealogy purposes. These methods may also be used to determine the likely geographic origin of a particular animal, species of animal etc. Populations are not necessarily geographic in nature. Thus, methods may also be used to identify the breed, species, kingdom, etc. of an organism. For example, the methods may be used to identify the particular species of dog or horse, e.g., for breeding, selling, or showing purposes.

Also encompassed are systems, apparatuses, kits, and machine-readable medium relating to such methods.

### BACKGROUND

For decades, scientists have known that geographical genetic diversity exists. People around the world share genetic traits with their neighbors that distinguish them from peoples living further away.

Traditional anthropology has classified four races corresponding to four major continents: African, European, Asian and American. This simple system of classification dates back to the 18^{th} century taxonomist Carolus Linnaeus and is still commonly used when describing ethnic groups and individuals. Certain areas of each continent are traditionally designated as pure representatives of each race, and other regions are assumed to be mixed between these presumably unmixed areas.

Early applications of genetic science used the traditional racial scheme in a “hand-me-down” fashion. The genetic differences between peoples traditionally identified as Black, White, Asian and Native American in North America are great enough to allow a rough estimate of an individual's “percentage” membership in each racial group. This approach has been used for medical and police applications as well as for individuals interested in learning more about their genetic ancestry. However, this racial scheme creates problems when used outside of the core regions ancestral to modern North Americans. Mankind cannot be described by a handful of 3-5 simplistic racial categories. Simplistic divisions of the world into 3-5 continents ignores important unique regions that do not neatly fall into presumed racial categories, such as North Africa, Polynesia or India.

For instance, a Pakistani or Samoan can be classified as some percentage of Native American, European, East Asian and Sub-Saharan Africa, but the resulting classification would be meaningless. At a theoretical level, this approach adds nothing to the popular or scientific understanding of human relationships and bestows an air of scientific legitimacy to outdated ideas of race. At a practical level, these theoretical limitations might have harmful consequences for example, for an individual administered a drug regimen based on a misleading percentage calculation. Clearly, the four-fold racial division provides an incomplete and misleading portrayal of the diversity of the human species.

Other genetic tests to determine ancestry include Y chromosome and mtDNA tests. However, while each person has thousands of ancestors, Y chromosome or mtDNA tests can only provide information about one lineage a person has inherited from one direct lineal ancestor.

### SUMMARY

The present inventors have invented methods of describing the genetic landscape of mankind by describing the world not as a stark checkerboard of racial divisions, but as a rich tapestry of overlapping world regions. The present methods objectively identify groups of populations based on neutral genetic markers. The result is a network of populations, such as world regions, each characterized by shared history and genetic patterns. Geographical outlines of these regions echo borders of countless empires, trade networks and kin groups.

As described further herein, the statistical methods developed and used by the present inventors, may be used for purposes other than identifying an individual's most likely ancestral geographic origin(s). By way of non-limiting example, methods, apparatuses, systems, machine readable medium, and kits may be adapted for uses such as identifying most likely geographic origin(s) of an individual person or animal (e.g., for law enforcement purposes); or for identifying a most likely breed(s) or species of animal.

Exemplary embodiments are generally directed to methods of determining an individual's relative likelihood of having a genetic match with one or more local populations, as compared to a generic index population. In particular, such methods may include determining a likelihood of the individual belonging to the at least one local population, e.g., by comparing genetic markers of the individual to the frequency of such markers occurring in at least one local population; determining a likelihood of the individual belonging to a generic index population, e.g., by comparing genetic markers of the individual to the frequency of such markers occurring in a generic index population; and comparing the likelihoods to determine the individual's relative likelihood of having a genetic match with the one or more local populations. The relative likelihoods with respect to each of several local populations may be ranked, if desired, to further demonstrate the likelihood of the individual matching each local population.

Example embodiments are also directed to apparatuses that include a server and software capable of performing methods herein or a portion thereof, such as determining a relative likelihood of an individual belonging to a local population as compared to a generic index population. Example embodiments are also directed to systems that include a server coupled to a database, where the database includes information regarding genetic markers occurring in at least one local population and/or in a generic index population.

Example embodiments are also directed to kits that include at least one device for determining genetic markers of an individual and a computer readable program product that includes a computer readable medium and a program capable of determining a relative likelihood of an individual belonging to a local population as compared to a generic index population.

Example embodiments are also generally directed to machine readable medium that include code segments or programs embodied on a medium that cause a machine to perform the present methods or any portion thereof.

### BRIEF DESCRIPTION OF THE DRAWINGS

Embodiments of the invention are herein described, by way of non-limiting example, with reference to the following accompanying drawings:

FIG. 1 illustrates approximate geographical boundaries of illustrative World Regions according to example embodiments;

FIG. 2 is a diagram illustrating relationships between the illustrative World Regions of FIG. 1 using statistical analysis;

FIG. 3 is a diagram illustrating relationships between the illustrative World Regions of FIG. 1 using statistical analysis;

FIG. 4 is an illustration of a composition of individual ethnic and national Native American populations as determined by example methods;

FIG. 5 is an illustration of a composition of individual ethnic and national African and Near Eastern populations as determined by example methods;

FIG. 6 is an illustration of a composition of individual ethnic and national European populations as determined by example methods;

FIG. 7 is an illustration of a composition of individual ethnic and national South Asian populations as determined by example methods;

FIG. 8 is an illustration of a composition of individual ethnic and national East Asian and Pacific populations as determined by example methods;

FIG. 9 depicts an example distribution of frequencies for a subset of a global population database at an example allele D8S1179 in accordance with example embodiments;

FIG. 10 depicts a sample individual genetic profile where genetic markers were determined at 13 alleles in accordance with example embodiments;

FIG. 11 is an illustration an example of partial matching results for a Basque individual, where the ten most likely matching populations, are ranked in order with the most likely matching population at the top;

FIGS. 12 and 13 illustrate Native Population Match results for the individual of FIG. 11 according to example embodiments, where FIG. 12 is a numerical illustration and FIG. 13 shows a relative numerical illustration on a world map;

FIGS. 14 and 15 illustrate Global Population Match results for the individual of FIGS. 11-13 according to example embodiments, where FIG. 14 is a numerical illustration and FIG. 15 shows a relative numerical illustration on a world map;

FIG. 16 illustrates numerical World Region Match results for the individual of FIGS. 11-15 according to example embodiments;

FIG. 17 depicts a genetic profile of an African individual, setting forth allele values at each of 13 loci in accordance with an example embodiment;

FIG. 18 is an illustration (both numerically and on a world map) of the top twenty Native population matches for the individual of FIG. 17 after performing a Native Population Match in accordance with example methods;

FIG. 19 is an illustration (both numerically and on a world map) of the top twenty Global population matches for the individual of FIG. 17 after performing a Global Population Match in accordance with example methods;

FIG. 20 is an illustration (both numerically and on a world map) of the top high resolution World Region matches for the individual of FIG. 17 after performing a World Region match in accordance with example methods;

FIG. 21 depicts a genetic profile of a European individual, setting forth allele values at each of 13 loci in accordance with an example embodiment;

FIG. 22 is an illustration (both numerically and on a world map) of the top twenty Native population matches for the individual of FIG. 21 after performing a Native Population Match in accordance with example methods;

FIG. 23 is an illustration (both numerically and on a world map) of the top twenty Global Population matches for the individual of FIG. 21 after performing a Global Population Match in accordance with example methods; and

FIG. 24 is an illustration (both numerically and on a world map) of the top high resolution World Region matches for the individual of FIG. 21 after performing a World Region match in accordance with example methods.

### DETAILED DESCRIPTION

The aspects, advantages and/or other features of example embodiments of the invention will become apparent in view of the following detailed description, which discloses various non-limiting embodiments of the invention. In describing example embodiments, specific terminology is employed for the sake of clarity. However, the embodiments are not intended to be limited to this specific terminology. It is to be understood that each specific element includes all technical equivalents that operate in a similar manner to accomplish a similar purpose.

It should be understood that example methods, apparatuses, systems, kits, and machine readable medium described herein may be adapted for many different purposes and are not intended to be limited to the specific example purposes set forth herein.

As used herein, “a” or “an” may mean one or more. As used herein, “another” may mean at least a second or more.

The terms “individual” and “organism” are used interchangeably herein and are intended to encompass an individual animal, including e.g., mammals (such as humans, dogs, horses, cats, etc.), and non-mammals. As used herein, the term “individual” is not limited to humans.

The term “population” is intended to encompass a grouping of more than one individual or organism. A “local population” is a grouping or subset of a larger population (“generic index population”) of individuals or organisms. A “local population” may, but does not necessarily, include a group of individuals from a similar geographic location (which may be referred to herein as a “World Region” or “World Region population”). Other “local populations” may include non-geographic groupings, such as groupings at a cladistic level. Non-limiting examples of “local populations” may include for example, towns, nations, ethnic groups, continents, species, subspecies, genus, family, order, class, phylum, or other grouping of individuals.

A “generic index population” is a grouping of more than one local population, which may be used for example, as a scaling population to which local population information is compared. By way of non-limiting example, a local population may be a nation within a generic index population of the world, or other geographic subsets and larger populations such as region/world, town or village/nation, nation/continent, etc. Local or generic populations may have boundaries that do not match nation or continent boundaries. Alternatively, the local to generic relationship may not be related to geography, such as a local population of a breed within a generic index population of a species, subspecies/species, species/genus, genus/family, family/kingdom, etc. Data regarding a “generic index population” may include for example an average, median or other formulation of data from all of the local populations making up the generic index population.

The term “genetic marker” is intended to encompass any portion of an individual's (organism's) genome that may be identified and compared to similar portions of the genome of a population of individuals. By way of non-limiting example, genetic markers may include a marker at any suitable genetic loci, such as allele values in the DNA at particular autosomal loci, or other genetic markers. Thus, genetic markers in an individual may be determined by sequencing the individual's allele values from a sample of the individual's DNA at N autosomal loci, where N is any positive integer. Standard forensic markers often used for paternity/maternity and other forensic DNA testing may be useful genetic markers for the present methods. Non-limiting examples of possible markers that may be used, include but are not limited to D3S 1358, TH01, D21S11, D18S51, D5S818, D13S317, D7S820, D16S539, CSF1PO, vWA, D8S1179, TPOX and FGA.

The determination of how many and which allele values and which loci are selected may vary depending many factors. For example, such factors may include what information is being sought, the availability of data with respect to a population to which the individual may be compared, and information regarding the uniqueness of allele values at particular loci. By way of non-limiting example, allele values may be sequenced at one or more short tandem repeat (STR) loci or single nucleotide polymorphism (SNP) loci. According to example embodiments allele values may be sequenced at at least 9 STR or SNP loci, or at 13 STR or SNP loci. STR loci are presently among the most informative polymorphic markers in the genome, but the invention is not intended to be limited in any way to markers at autosomal STR loci.

The term “match” as used herein is not intended to denote an exact match, but rather an indication of the most likely genetic match between an individual and a population, based on statistical methods. For example, an individual may be designated herein as matching a population based on their relative likelihood of matching that population (as compared to a generic index population) being greater than the relative likelihood of “matching” one or more other populations. A match with a particular ethnic or national population sample does not guarantee that the individual or a recent ancestor (parent or grandparent, for instance) are a member of that population (e.g., ethnic group). However, a match may indicate for example, a population where the individual's combination of ancestry is common, which is most often due to shared ancestry with that population.

Example embodiments are generally directed to methods of determining an individual's (including mammals such as humans, or other animals) relative likelihood of having a genetic match with one or more local populations, as compared to a generic index population. In particular, examples of such methods may include determining a genetic likelihood of the individual belonging to at least one local population (e.g., by comparing genetic markers of the organism to the frequency of such markers occurring in at least one local population); determining a genetic likelihood of the individual belonging to a generic index population (e.g., by comparing genetic markers of the organism to the frequency of such markers occurring in a generic index population); and comparing the likelihood of the individual belonging to the at least one local population to the likelihood of the individual belonging to the generic index population to determine the individual's relative likelihood of a genetic match with the one or more local populations.

According to example embodiments, methods of the invention may be used to identify the most likely geographic origin of an individual's ancestors. Such uses may be desirable for example for genealogy purposes. Thus, the likelihood of an individual human belonging to (e.g., having ancestors from) one geographic local population (also referred to as a “World Region”) may be calculated and compared to the likelihood of that individual belonging to a generic world index population that includes a plurality of geographical local populations.

The methods herein may also be used for purposes other than identifying an individual's most likely ancestral geographic origin(s). For example, in addition to identifying an individual's most likely ancestral geographic origin(s), methods, apparatuses, systems, machine readable medium, and kits may be adapted for uses such as: identifying most likely geographic origin(s) of an individual themselves; identifying most likely geographic origin(s) of an animal; and identifying most likely breed(s) or species of animal. These uses are non-limiting examples of some of the many possible embodiments.

According to example embodiments, methods of the invention may be used to identify an individual's most likely geographic origin. Such uses may be desirable for law enforcement purposes. For example, if a DNA sample is left behind at a crime scene, it may be possible to determine information about the most likely national/regional origins of the individual whose DNA was at the crime scene and analyzed.

As indicated above, other example methods may be used to identify a breed, species, kingdom, etc. of an organism. By way of non-limiting example, methods may be used to identify the particular breed of dog or horse, which may be useful e.g., for breeding, selling, and/or showing purposes. Thus, example embodiments may include calculating the relative likelihood of an individual animal (such as a dog or horse) belonging to one breed as compared to the likelihood of that animal belonging to an index population of the species. Other example embodiments, involving non-humans, may include using the methods herein to determine a likely geographic origin of a particular animal (or its ancestors), species of animal, etc.

Example embodiments include determining a genetic likelihood of an individual belonging to at least one local population. Example methods of determining the likelihood of an individual belonging to at least one local population may include comparing one or more genetic markers present in the individual (e.g., at a plurality of genetic loci) to the frequency of such genetic markers occurring in the at least one local population. Genetic markers in the individual may be determined for example, by sequencing the individual's allele values from a sample of the individual's DNA at N autosomal loci, wherein N is any positive integer. The autosomal loci may be for example, STR loci or SNP loci, but are not limited to such.

The likelihood of the individual belonging to at least one local population may be determined for example, by a method that includes extracting from a database, frequencies p matching the individual's allele value at each locus w, w=1 . . . 2N, where N is the number of genetic loci for which data is collected from the individual, for each local population; and determining a joint probability Pⱼ of an individual matching a local population j by multiplying the extracted frequencies p_{w\j} using the following formula

$${P}_{j} = {\prod }_{w = 1}^{2 N} {p}_{w \vert j}$$

According to example embodiments, the joint probability Pⱼ may be adjusted for confidence. By way of non-limiting example, the joint probability Pⱼ of an individual matching a local population j, may be adjusted by determining a lower bound of a confidence interval to arrive at a joint matching probability \tilde{P}ⱼ (also referred to herein as match likelihood or likelihood). \tilde{P}ⱼ may be determined for example by a method using the following formula:

$${\tilde{P}}_{j} = exp \{ log {P}_{j} - {Z}_{C} \sqrt{\frac{1}{{n}_{j}} {\sum }_{w = 1}^{2 N} \frac{1 - {p}_{w \vert j}}{{p}_{w \vert j}}} \}$$

wherein p_{w\j} is a frequency of the individual's allele value at each locus w in population j, w=1 . . . 2N, where N is the number of genetic loci for which data are collected from the individual, nⱼ is the number of individuals in population j for which genetic data were collected, and Z_{C} is a z-score corresponding to the C confidence level.

According to example embodiments, a local population may be defined by a method that includes using any multivariate clustering algorithm (such as K-means) to divide data from a set of population samples into groups. For example, the larger database of populations may be separated into K groups. Genetic marker (e.g., allele) frequencies for a World Region K can be for example, a median, mean or any other general combination of genetic marker frequencies of local populations in group K. This local World Region population may be compared to a generic index population as described further below. By way of non-limiting example, representative populations for World Regions may be obtained using a K-means analysis of all populations in a global database. This analysis may identify major divisions in global genetic variation corresponding to major continental regions (e.g., European, Sub-Saharan African, East and South Asian, and Native American). Representative populations for each of these World Regions may be chosen by their proximity to cluster centers. These representatives are used as reference points for the clusters, to which individuals are compared to estimate their continental ancestry. According to example embodiments, World Regions may be determined by median, means or other statistical methods.

Thus, various local populations (such as World Regions) may be identified by objective mathematical criteria and information regarding such populations may be maintained in a database to be used for determining an individual's most likely genetic matches to such populations. According to example embodiments many of these World Regions may correspond to cultural or linguistic groups. For instance, Slavic-speaking peoples share a predominance of the Eastern European region. Other World Regions cross national and cultural boundaries as they exist today. For instance, the Asia Minor region can be found from modern day Southern Italy to Turkey to Afghanistan, and includes speakers of Indo-European, Afro-Semitic, Altaic and Indo-Iranian languages.

According to example embodiments, there may be occasions where one or more of an individual's allele values are not used in calculating matches. In particular, there exist numerous allele values at each particular locus Z, that are not informative when calculating matches. Let pⱼ denote the proportion of individuals having specific allele value z in population j. An allele “z” may be identified as a “weak allele,” and therefore according to certain embodiments may not be used in the calculations and methods herein, if it fails certain mathematical criteria. By way of non-limiting example, a particular weak allele may not be used in the calculations herein if the allele fails both of the following criteria:
a) pₘₐₓ/p₉₅<3, where pₘₐₓ is the maximum frequency observed in all populations at allele z of locus Z and p₉₅ is the 95% percentile value of the frequencies.
b) at least 90% of the top 20 populations with the highest pⱼ values are in at most two World Regions.
   An example of a weak allele, that is, an allele failing both criteria is provided below. It should be noted that the exact criteria used to define a weak allele, may vary within the scope of these embodiments.

According to example embodiments, when a particular allele occurs in an individual, but is not observed in a population sample, a very low allele frequency (such as 0.001) may be imputed, so as to err on the side of over-exclusiveness. This is in contrast to methods used in standard paternity match analysis and other forensic identity analysis methods, where match calculations aim to err on the side of over-inclusiveness. For example, in other methods, when a particular allele is not observed in a population, a minimum value is typically imputed according to a standard formula, so that a frequency of zero is not used in calculations.

Example embodiments of the methods herein include determining a genetic likelihood of an individual belonging to a generic index population. Example methods of determining the likelihood of an individual belonging to a generic index population may include comparing one or more genetic markers present in the individual (e.g., at a plurality of genetic loci) to the frequency of such genetic markers occurring in the generic index population.

According to example embodiments, the likelihood of the individual belonging to a generic index population may be determined by a method that includes extracting from a database, frequencies p matching the individual's allele value at each locus w, w=1 . . . 2N, where N is the number of genetic loci for which data is collected from the individual, for the generic index population GI (also referred to herein as a generic human index or GHI in the case where the individual is a human); and determining a joint probability P_{GI} of an individual matching the generic index population by multiplying the extracted frequencies p_{w\GI} using the following formula

$${P}_{GI} = {\prod }_{w = 1}^{2 N} {p}_{w \vert GI} .$$

According to example embodiments, the joint probability P_{GI} may be adjusted for confidence. By way of non-limiting example, the joint probability P_{GI} of an individual matching a generic index population may be adjusted by determining a lower bound of a confidence interval to arrive at a joint matching probability \tilde{P}_{GI}. \tilde{P}_{GI} may be determined for example by a method using the following formula:

$${\tilde{P}}_{GI} = exp \{ log {P}_{GI} - {Z}_{C} \sqrt{\frac{1}{{n}_{GI}} {\sum }_{w = 1}^{2 N} \frac{1 - {p}_{w \vert GI}}{{p}_{w \vert GI}}} \}$$

P_{GI} is the joint probability of an individual matching the generic index population, p_{w\GI} is a frequency of matching the individual's allele value at each locus w, w=1 . . . 2N, and N is the number of genetic loci for which data is collected from the individual. n_{GI} may be determined by the following formula:

$${n}_{GI} = \frac{1}{K} {\sum }_{j = 1}^{K} {n}_{j}$$

where K is a number of local populations used to calculate the generic index population, and nⱼ is a number of individuals comprising local population j.

The frequency of genetic markers occurring in a generic index population may be determined for example, by determining frequencies of alleles occurring at each of N loci for multiple local populations and averaging or determining the median of frequencies for each allele for all of the multiple local populations. According to non-limiting example embodiments, the local population may be a World Region population and the generic index population is an average or median of all World Region populations.

As indicated above, a local population may be a nation within a generic index population of the world, or other geographic subsets and larger populations such as region/world, town or village/nation, nation/continent, etc. Local or generic populations may have boundaries that do not match nation or continent boundaries. Alternatively, the local to generic relationship may not be related to geography, such as a local population of a breed within a generic index population of a species, subspecies/species, species/genus, genus/family, family/kingdom, etc. For example, a generic index population may be selected from the group consisting of a kingdom, phylum, class, order, family, genus, species, and any subdivisions thereof. Thus, by way of example, each local population may be a breed of organisms, and the generic index population may be a species of organisms. Further, the individual may be an individual dog, where each local population is a breed of dogs, and the generic index population is dogs.

The GI (or GHI) is a fixed reference point to which all individual matches with actual populations are measured and serves as the “null hypothesis” for each match that the individual's genetic profile is “generic” rather than indicative of e.g., regional or ethnic genetic affiliation. As more data becomes available for one or more local populations making up the global index population, for example if a new set of data is obtained for a new native tribe or individual data is added to known local populations, the GI data may be recalculated. When this occurs, methods herein may be repeated to provide updated likelihood calculations.

Example embodiments may further include comparing the likelihood of the individual belonging to at least one local population to the likelihood of the individual belonging to a generic index population. The methods of comparison may include for example, comparing joint probabilities or joint matching probabilities (adjusted for confidence). Methods of calculating the joint probabilities, whether or not the probabilities are adjusted for confidence, and/or how they are adjusted may vary within the scope of the present methods.

Example embodiments of such comparisons may include dividing the likelihood of the individual belonging to a first local population by the likelihood of the individual belonging to a generic index population to determine a relative likelihood ratio of the individual belonging to the local population. It is contemplated that methods within the scope of this application of comparing the probability of an individual matching a local population to the probability of that individual matching a generic index population, may include methods other than pure division.

According to example embodiments, a relative likelihood ratio LR (or match likelihood index (MLI) score) of an individual belonging to a local population as compared to a generic population may calculated using the following formula:
LR=\tilde{P}ⱼ/\tilde{P}_{GI},
wherein \tilde{P}ⱼ is a joint probability of an individual matching a local population j, adjusted for confidence; and \tilde{P}_{GI} is a joint probability of an individual matching a global index population GI, adjusted for confidence.

Example embodiments may include comparing the likelihood of the individual belonging to a second or more local population(s) to the likelihood of the individual belonging to a generic index population to determine relative likelihood ratios of the individual belonging to each of the second or more local populations. Thus, several relative likelihood ratios may be obtained for each of several local populations. In such methods, the relative likelihood ratios of the individual belonging to each of several local populations may be ranked or otherwise denoted. Ranking or comparing more than one relative likelihood ratio may assist in demonstrating the likelihood of the individual matching each local population. For example, such rankings may include a numerical ranking with the local population having the highest relative likelihood ratio being first or last in a list. Such a list may include for example, the top ten or top twenty matching populations. Other methods of denoting the relative likelihood ratios may include color coding (e.g., on a map or on a chart of breeds); or any indication that would allow one (by sight, sound, feel (e.g., Braille), etc) to be able to determine which local population(s) are more likely a match to the individual than other local population(s).

According to other example embodiments, the relative likelihood ratios of the individual belonging to each of several local populations, may be numerically compared to one another. For instance, the most likely genetic matches may be presented for example with a match likelihood index (MLI) score. If the top ranked match MLI or LR for an individual is 30, and the second ranked match MLI is 15, one can divide 30/15 to see the relative likelihoods between those matches.

Multiple forms of analysis may be performed on an individual to determine possible matches to various local populations. For example, where information is sought regarding an individual's most likely ethnic and geographical origin, a combination of methods, such as a Global Population Match, Native Population Match, and/or World Region Match (described further herein), may present an ethnically and geographically specific indication of the individual's most likely origin. It should be noted that such Global, Native and World Region matches are non-limiting examples of some of the many possible methods that may be performed.

According to example embodiments, methods may include a Global Population Match to determine an individual's most likely genetic match to global (local) populations, including both native ethnic groups (discussed further below) and modern Diaspora and admixed populations. As used herein, the term “Global population” may include for example, all population samples in a database. The most likely genetic matches may then be presented for example by an MLI score for each. Such information regarding populations to which the individual most likely matches, whether expressed by MLI score or other measure of likelihood, may then be presented for example, to the individual. An example method of how such information may be presented may include plotting the locations of the Global populations that are the most likely matches on a map. Points and/or shading and/or coloring on the map to indicate locations of most likely matches may further include an indication of the magnitude of the likelihood of a match. For example, matches having the highest MLI score may be darkest, or a particular color, or scored in a certain manner, where a key may be provided to inform the reader of the meaning of whatever indication is provided.

Non-limiting examples of Modern Diaspora ethnic groups may include African-Americans, European-Americans or Asian-Americans. Modern Diaspora populations may be descended from immigrants who have recently moved from their homelands to live around the world, often blending with other peoples. Population matches may be divided between Global and Native to identify Diaspora affiliations as well as genetic links to indigenous peoples. For instance, African-Americans may match African Diaspora populations such as African-Americans from various U.S. States, Afro-Brazilians and related peoples. However, their Native Population Match results can also indicate roots in indigenous African, European or Native American populations.

According to example embodiments, methods may include a Native Population Match to determine an individual's most likely genetic match with a subset of Global populations identified as Native. The term “Native Populations” as used herein is intended to encompass those that have experienced minimal admixture for example, within the past 500 years or so. The amount of admixture and/or the number of years over which such admixture has occurred may vary. But the idea is that a match may be performed specifically directed toward populations that have experienced significantly less admixture in recent years than other populations. Minimal admixture may be for example, 20% or less, 10% or less, or 5% or less over the last 100, 200, 300, 400, 500 or more years. These methods are intended to try to exclude e.g., variations in world population data caused by significant admixture of populations in recent years in certain populations, for example in the U.S. or Canada having a high degree of admixture. By way of non-limiting example, Native populations may include Native Amazonians, Scottish, Egyptians or Japanese. As with Global Population Match results, the most likely genetic matches in a Native Population Match may then be presented with by their match likelihood index (MLI) scores. For instance, a Native Population Match with Macedonia having an MLI score of 45.2 indicates that the individual's genetic ancestry is 45.2 times as likely in Macedonia as in the generic population (e.g., the world). Such information regarding native populations to which the individual most likely matches, whether expressed by MLI score or other measure of likelihood, may then be presented for example, to the individual, by use of a map or otherwise as discussed throughout this application.

According to example embodiments, methods may include a World Region match to determine an individual's most likely genetic match with World Regions. World Regions may be major biogeographic clusters or subdivisions of human genetic diversity or may be determined using medians or means of multiple member populations, rather than a “cluster representative.” World Region match results may indicate an individual's most likely continent(s) of origin, and can indicate whether an individual is of mixed or relatively unmixed continental ancestry. As with other types of match results, the most likely genetic matches in a World Region match may then be presented with a match likelihood index (MLI) score. Such information regarding native populations to which the individual most likely matches, whether expressed by MLI score or other measure of likelihood, may then be presented for example, to the individual, by use of a map or otherwise as discussed throughout this application.

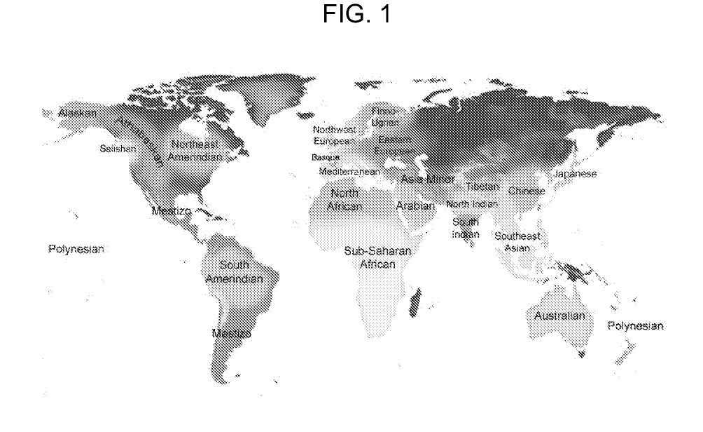

According to example embodiments, the present methods may provide a likelihood of an autosomal (e.g., STR or SNP) DNA profile of an individual in several (e.g., 23) World Regions. World Regions may be compared to individual populations to assist in determining an individual's most likely region of ancestry. The map depicted at FIG. 1 illustrates approximate geographical boundaries of example World Regions in accordance with example embodiments. Even within the borders of regions, individuals can be found with genetic ties to neighboring and sometimes distant regions. As shown in FIG. 1, World Regions may include for example the following regions:

Native American:
Arctic: Inuit peoples of Alaska.
Athabaskan: Athabaskan speaking peoples of Western North America.
Great Plains: Native peoples of the Great Plains of North America.
Salishan: Salish speaking peoples of the American Pacific Northwest.
South Amerindian: Native peoples of Central and South America.
Ojibwa (not shown in FIG. 1): Sub-arctic Northern Canada.
Mestizo: Native Americans mixed with Europeans and Africans.

European:
Eastern European: The Slavic speaking region of Eastern Europe.
Finno-Ugrian: The Uralic speaking region of Northeastern Europe.
Mediterranean: The Romance speaking region of Southern Europe.
Northwest European: The Celtic and Germanic speaking region of Northwestern Europe.

African and Near Eastern:
Arabian: The Arabian Peninsula.
Asia Minor: The East Mediterranean and Anatolia to the Tarim Basin.
India: Subcontinental India.
India Tribal (not shown): Tribal peoples of eastern India.
North African: North Africa.
North India: North Subcontinental India.
Sub-Saharan African: Africa south of the Sahara Desert.

Asia/Pacific:
Australian: Aboriginal peoples of Australia.
Chinese: The Chinese region of East Asia.
Japanese: The Japanese Archipelago.
Polynesian: The Polynesian Islands.
Southeast Asian: Southeast Asia and the Malay Archipelago.
Tibetan: The Himalayas and Tibetan Plateau.
Timorese: East Timor.

Rather than relying on presumed racial or ethnic divisions, the inventors have defined World Regions by objective mathematical criteria. In particular, World Regions have been identified by the present inventors using statistical analysis of a global DNA database of over 500 modern population samples around the world to identify groups of populations with shared genetic characteristics. These genetic groups may then be plotted on a map and named according to the geographical regions they occupy. It should be understood that as more data become available regarding population samples, and new population samples become available, World Regions may be updated and change. Such changes may include for example, changing of boundaries and/or names of regions within boundaries, or the addition or deletion of previously defined World Regions.

Each World Region represents a unique genetic family within the human species shaped by shared history and geography. Each region is characterized by a distinctive pattern of allele frequencies across the genetic loci studied. Although all humans are connected by ancient common origins, each of these genetic families shares a unique relationship due to more intense and persistent contacts within a geographical area. The present inventors have developed methods to distinguish these genetic families without relying on presumed racial or ethnic categories.

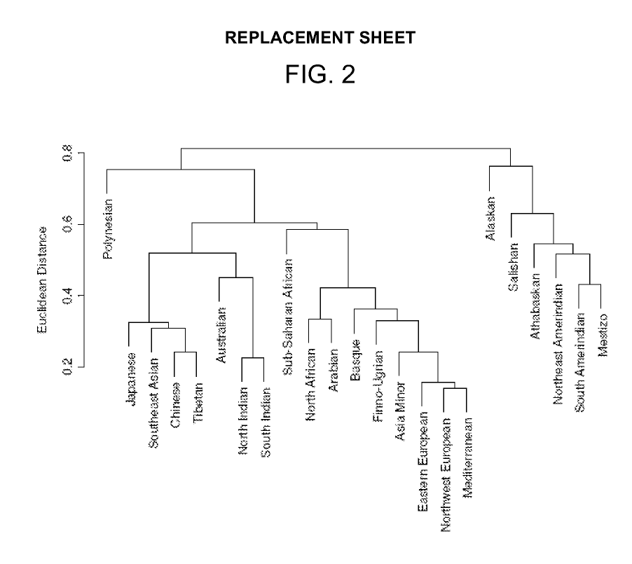

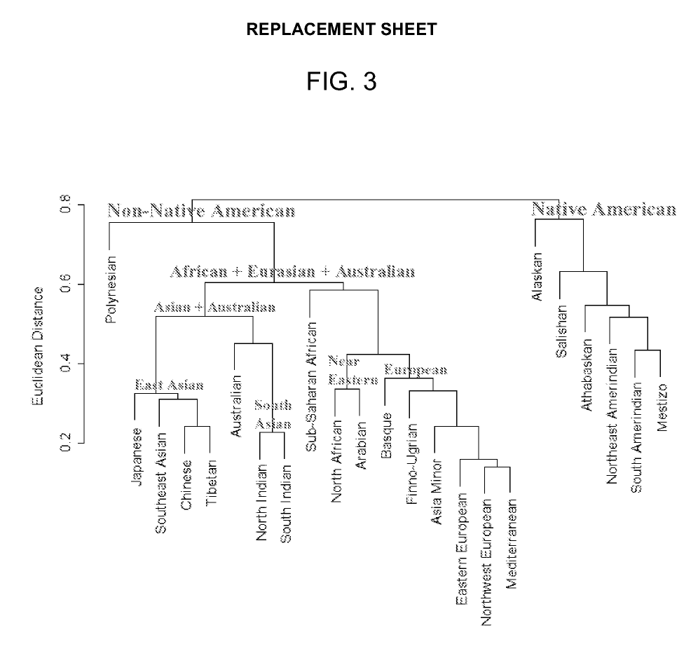

Hierarchical clustering may be performed on the twenty three World Region clusters with the distance metric as the sum of absolute differences. In the plots depicted at FIGS. 2 and 3, the distance between clusters is the average of the distances between the points in one cluster and the points in the other cluster. FIG. 2 depicts generally how example World Regions are related.

FIG. 3 illustrates the relationships between example World Regions identified by the inventors using statistical analysis. Closely related regions appear towards the bottom of the diagram. For instance, the Northwest European and Mediterranean regions are the two most closely related of these 23 regions. The deepest divisions appear at the top (root) of the tree. For instance, the Polynesian region is only distantly related to other World Regions and branches off alone towards the top of the tree diagram. Individual regions group together to form families and super-families of regions. Most of these larger groupings correspond to major continents. For instance, all four East Asian regions (Japanese, Southeast Asian, Chinese and Tibetan) form their own family. This East Asian family is part of a larger Asian super-family that also includes South Asian (North Indian and South Indian) and Australian regions. Similarly, all six Native American regions are part of their own super-family that is distinct from the other super-family that includes all Asian, Pacific, European and African regions.

The relationships illustrated by FIG. 3 are the cumulative product of for example, (1) genetic contact within each region created by migrations, intermarriage, and gradual diffusion; and (2) relative isolation from other regions. Natural features that make these contacts easier or more difficult have a strong effect on regional relationships. Such natural features may include for example: waterways, mountain regions, fertile plains, and continental borders shape the pathways of human interactions that create both cultural areas and genetic regions. For instance, the historical difficulty of travel between Asia and North America corresponds to the great distance between the Native American super-family and all other regions.

Further example methods may include implementing any of the present methods in an admixture analysis. A major flaw of present admixture testing is that it assumes a given individual is descended from presumed population references (usually representing standard racial categories). This creates errors when an individual is not, in fact, descended from these presumed sources of admixture. According to example embodiments, an individual's substantial match scores according to the present methods may be used to determine an admixture. For example, an individual's substantial match scores e.g., with World Regions, may be identified by a likelihood comparison. Then all World Regions for which the individual obtains a substantial likelihood score (e.g., greater than 1.0, or greater than the generic index) may be used as presumed sources of admixture in an admixture estimate. This eliminates the use of spurious admixture source populations not related to that individual. Thus, a World Region analysis can be used as one tier of a two-tiered admixture analysis.

Example embodiments are also directed to apparatuses that may include a server and software capable of performing methods herein. By way of non-limiting example, software may be capable of determining a first likelihood of an individual belonging to a local population by comparing genetic markers present in the individual to a frequency of such genetic markers occurring in the local population; and determining a second likelihood of the individual belonging to a generic index population by comparing the genetic markers present in the individual to a frequency of such genetic markers occurring in the generic index population. The software may be capable of comparing the first likelihood to the second likelihood. The software may be further be capable of determining a relative likelihood of the individual belonging to the local population as compared to the generic index population. Information regarding the frequency of genetic markers occurring in each population may be accessed by the server by various methods. The information may be stored in one or more databases that may be accessed separately, such as over the internet, or in a database coupled to the server (as in the systems described below).

Example embodiments also include systems that include a server coupled to a database. The database may include information regarding genetic markers occurring in at least one local population and/or in a generic index population. Information regarding genetic markers occurring in a generic index population might be a separate component of the database that also includes information regarding genetic markers occurring in at least one local population, or may be information derived from the information regarding the local population(s). As with other embodiments, in example embodiments, the server may include software capable of performing the methods herein, or a portion of such methods. For example, such software may be capable of determining a first likelihood of the individual belonging to a local population by comparing genetic markers present in the individual to a frequency of such genetic markers occurring in the local population; and determining a second likelihood of the individual belonging to a generic index population by comparing the genetic markers present in the individual to a frequency of such genetic markers occurring in the generic index population. The software may be further capable of comparing the first likelihood to the second likelihood.

Example embodiments are also generally directed to machine readable medium (such as a computer readable medium) that include code segments embodied on a medium that, when read by a machine, cause the machine to perform any of the present methods or portions thereof. Thus, example embodiments of a machine readable medium may include executable instructions to cause a device to perform one or more of the present methods or portions thereof.

Example embodiments also include computer-readable program products that include computer-readable medium and a program for performing one or more of the present methods or portions thereof.

A medium (such as a machine-readable medium or computer-readable medium) may include any medium capable of storing data that can be accessed by sensing device such as a computer or other machine. A machine-readable medium includes servers, networks or other medium that may be used for example in transferring code or programs from computer to computer or over the internet, as well as physical machine-readable medium that may be used for example, in storing and/or transferring code or programs. Physical machine-readable medium includes for example, disks (e.g., magnetic or optical), cards, tapes, drums, punched cards, barcodes, and magnetic ink characters and other physical medium that may be used for example in storing and/or transferring code or programs.

Example embodiments are also directed to kits that include at least one device for determining genetic markers of an individual and a machine readable medium that includes a medium and a program capable of determining a relative likelihood of an individual belonging to a local population as compared to a generic index population.

Example devices for determining genetic markers of an individual may include at for example, a sample collector (such as a swab capable of collecting DNA). Other example devices may include a device capable of reading DNA from a sample collector, such as a device into which a swab may be inserted.

The following examples illustrate non-limiting embodiments. The examples set forth herein are meant to be illustrative and should not in any way serve to limit the scope of the claims. As would be apparent to skilled artisans, various changes and modifications are possible and are contemplated and may be made by persons skilled in the art.

### EXAMPLE 1

In this example, observed allele frequency data was used to simulate 4,000 individual genetic profiles for studied world populations. Each simulated profile was processed using the present methods, and in particular using an algorithm, which measured the simulated individual's occurrence frequency in each of 23 World Regions. The strongest regional match was then identified for each simulated individual. These primary matches were then tallied for all simulated profiles to produce regional affiliation proportions.

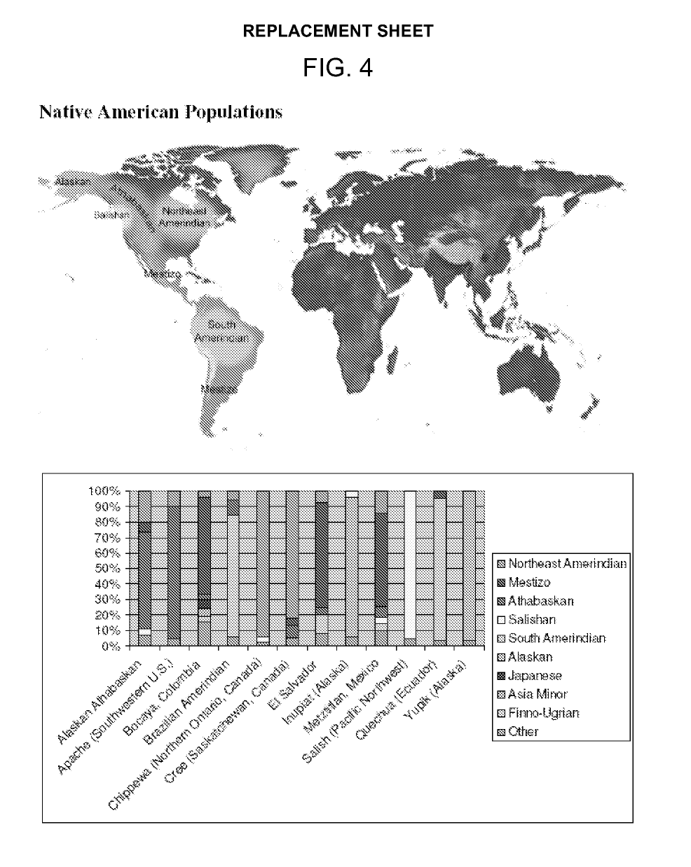

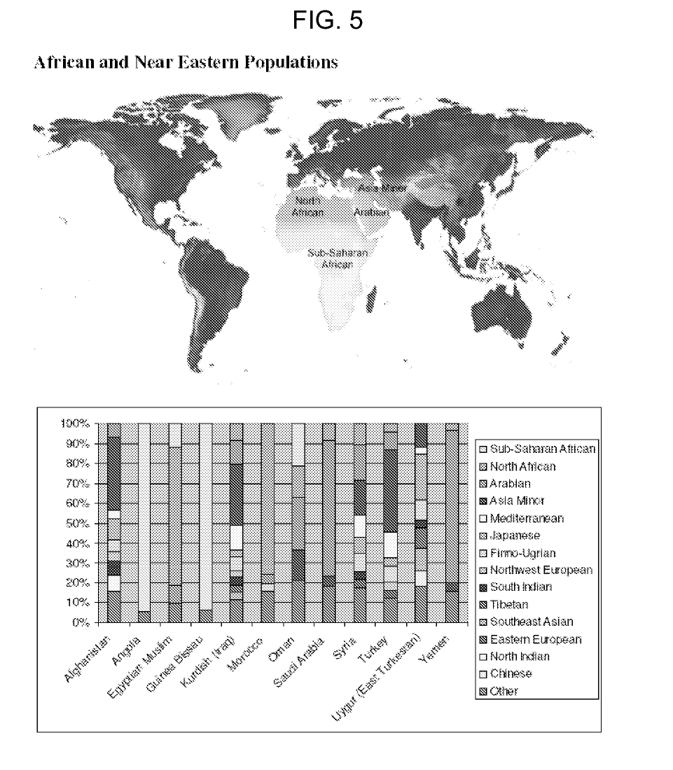

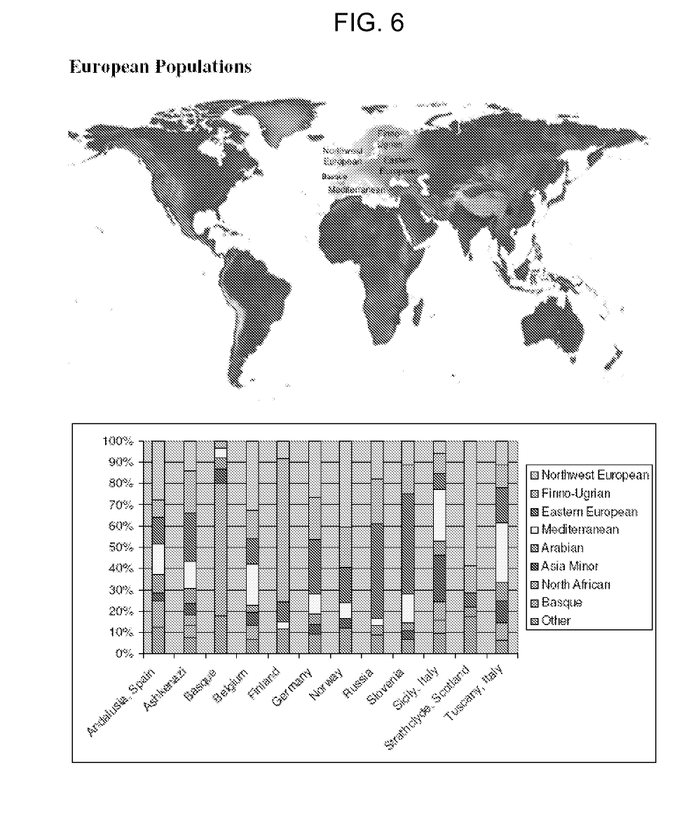

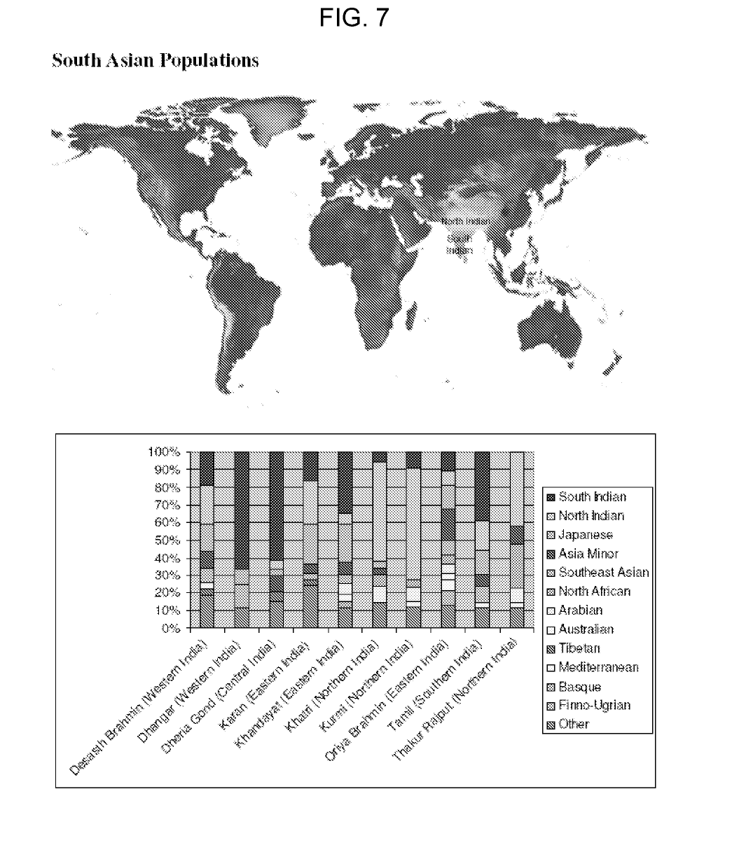

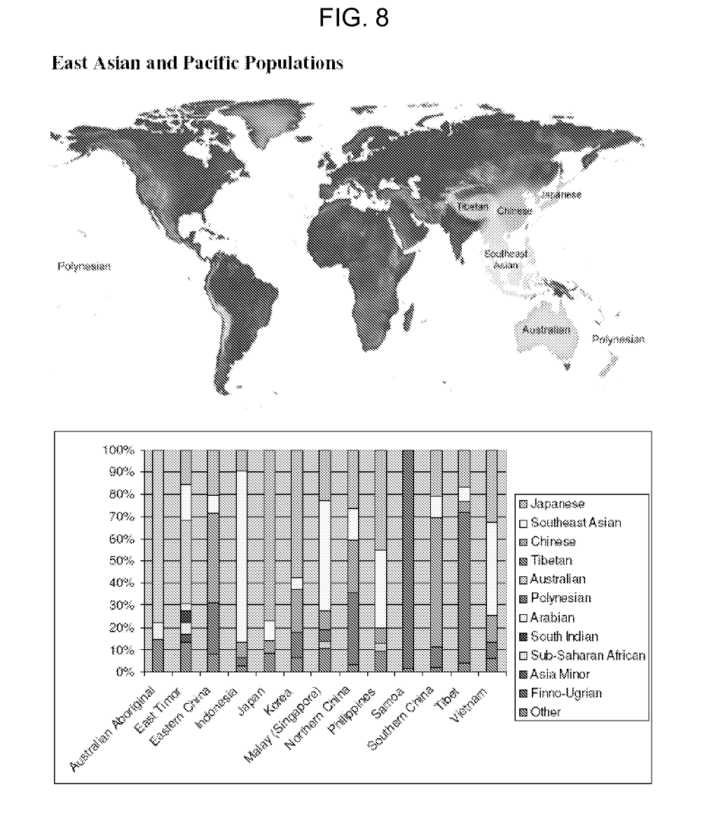

The individual populations include a spectrum of regional affinities. This study (the results of which are depicted in FIGS. 4-8) illustrates the composition of individual ethnic and national populations. FIG. 4 illustrates Native American populations. FIG. 5 illustrates African and Near Eastern populations. FIG. 6 illustrates European populations. FIG. 7 illustrates South Asian populations. FIG. 8 illustrates East Asian and Pacific populations. As shown in FIG. 4, based on the results of this study, approximately 63% of Alaskan Athabaskans belong primarily to the Athabaskan World Region that also includes Apache and Navajo of the Southwestern United States.

As indicated above, as more data is incorporated and a statistical analysis is refined, a map of twenty three example World Regions may be clarified and refined. However, a number of basic points have become apparent as a result of inter alia, this study. First, Native Americans, traditionally considered a homogeneous group or perhaps a minor offshoot of the Asian “Mongolian race,” are instead a complex group of World Regions. The genetic divisions between some of these regions are deeper than those between traditional races. For instance, the distance between Salishans and all other non-Alaskan Native Americans is greater than the distance between Europeans and Asians (see tree diagram of FIG. 2).

Second, intermediate regions within Eurasia are not equivalent to simple admixtures between far Western Europeans and far Eastern Asians. Analysis of non-coding regions indicates Turks, Tibetans, North Indians and others possess unique genetic characteristics omitted by a simple racial admixture model. South Asia is the home of at least two unique World Regions not consistent with a simple model of East-West contact. The North and South Indian regions are each characterized by distinct allele frequencies, suggesting each of these places has become a unique genetic homeland rather than only a recipient of migrations. Genetic ties to distant Australia suggest this South Asian cradle is ancient and might house genetic diversity not yet described by existing population studies. In Europe, previous studies have demonstrated geographical structure within Italy, with extremes in the north and the south.

Studies and analysis by the inventors confirm that the northern parts of Italy are home to a “Mediterranean” regional group and connections to continental Europe. Further south, regional affiliations with the northern parts of Europe decrease while connections to an “Asia Minor” region become more prominent. This Asia Minor region is most typical of peoples living in Anatolia and Mesopotamia and extending as far east as Central Asia (the Turkic speaking Uygurs of East Turkestan). Polynesia remains an outlier region not clearly connected to any others. This could be due to geographic isolation in the Pacific Ocean. However, Australia retains genetic connections to Asia and forms a group with the two Subcontinental Indian regions. East Timor retains significant links to Australia as well as Southeast Asia.

### EXAMPLE 2

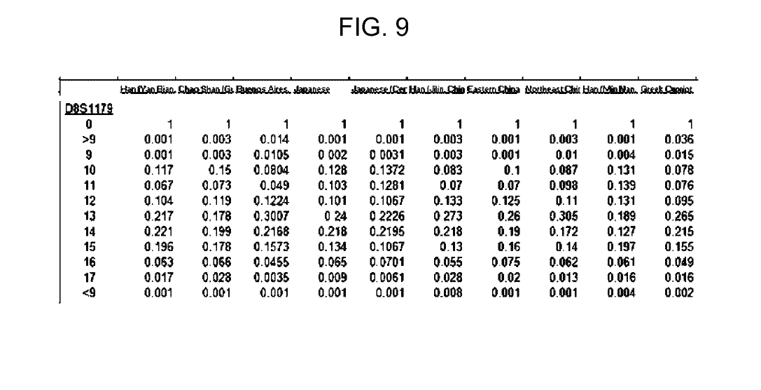

In this example, a Global Population database is used containing N (in this case 280) populations, each including a varying number of individuals. Population data is extracted from studies published in academic journals, including sources such as forensic science journals, and assembled with standard spreadsheet software. For each population j, the frequency p of individuals having a certain allele value at 13 STR loci was recorded. FIG. 9 shows an example distribution of frequencies for a subset of the Global Population database at the allele D8S1179.

World Regions are identified as follows: a standard K-means clustering algorithm was used to separate all the populations in the Global Population database into k=4 distinct clusters (groups). These clusters correspond to major continental regions (European, Sub-Saharan African, East and South Asian, and Native American). For each group k, a single population with the smallest Euclidian distance measure to the cluster's centers may be selected as representative of the group. Each of these four representative populations may be used as a reference point for the entire cluster, to which individuals are compared to estimate their continental ancestry for the World Region Match portion of analysis, as described below.

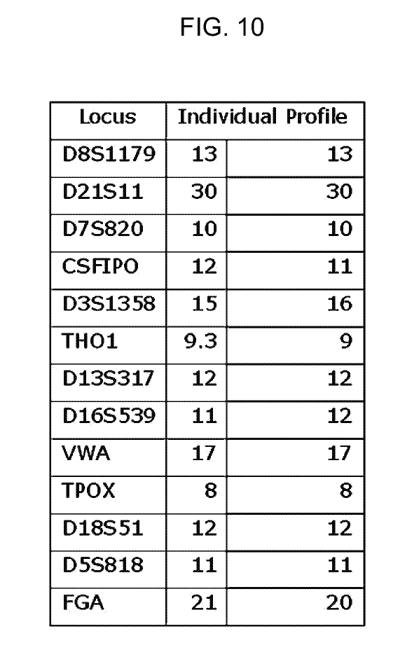

According to this example, genetic information is collected from an individual as follows: an autosomal STR profile is obtained for an individual, by collecting DNA from the individual using a standard cheek swab and his/her allele values at 13 autosomal STR loci, including D8S1179, D21S11, D7S820, CSFIPO, D3S1358, THO1, D13S317, D16S539, VWA, TPOX, D18S51, D5S818, and FGA are sequenced. For each individual, there are a total of 26 values, as the individual receives a unique allele from each parent at each locus. A sample individual genetic profile is shown in FIG. 10. Depending on the method being implemented all or some of these markers may be implemented. For example, values from nine of these markers may be used to compute Native and Global population matches, while values from all thirteen markers may be used to compute high resolution World Region matches.

Next, a GeoGenetic match for an individual may be produced by executing the following algorithm:
Step 1: For each population j, the frequencies matching the individual's allele value at each locus w, w=1 . . . 26 (where 26 is 2N and N is the number of autosomal loci), are extracted from the database. Then, the joint probability Pj of an individual matching jth population is computed by multiplying the extracted proportions, as follows:

$${P}_{j} = {\prod }_{w = 1}^{2 N} {p}_{w \vert j}$$
wherein p_{w\j} is a frequency of the individual's allele value at each locus w in population j, w=1 . . . 2N, where N is the number of genetic loci for which data are collected from the individual.
Step 2: To account for sample size variation among populations, 95% confidence intervals (CI) for the joint probability that an individual belongs to a population j are obtained using the delta method. Then, the lower bound of this CI (denoted by tilde) is taken as a joint matching probability instead, as follows:

$${\tilde{P}}_{j} = exp \{ log {P}_{j} - {Z}_{C} \sqrt{\frac{1}{{n}_{j}} {\sum }_{w = 1}^{2 N} \frac{1 - {p}_{w \vert j}}{{p}_{w \vert j}}} \}$$
wherein nⱼ is the number of individuals in population j for which genetic data were collected, and Z_{C} is a z-score corresponding to the C confidence level.
Step 3: To make the interpretation of the lower bound of the 95% CI for all j meaningful, a synthetic Generic Human Index (GHI or GI) population is produced. This is done by averaging the frequencies for each specific allele for all populations and assuming that the sample size for GI population is the average of all population sample sizes, as follows:

$${P}_{GI} = {\prod }_{w = 1}^{2 N} {p}_{w \vert GL}$$
where p_{w\GI} is a frequency of matching the individual's allele value at each locus w, w=1 . . . 2N, and N is the number of genetic loci for which data is collected from the individual. The lower bound of the 95% CI for the joint probability that an individual belongs to the GI population is calculated as follows:

$${\tilde{P}}_{GI} = exp \{ log {P}_{GI} - {Z}_{C} \sqrt{\frac{1}{{n}_{GI}} {\sum }_{w = 1}^{2 N} \frac{1 - {p}_{w \vert GI}}{{p}_{w \vert GI}}} \}$$
where n_{GI} may be determined by the following formula:

$${n}_{GI} = \frac{1}{K} {\sum }_{j = 1}^{K} {n}_{j}$$
where K is a number of local populations used to calculate the generic index population, and nⱼ is a number of individuals comprising local population j.
Step 4: A Match Likelihood Index (MLI or LR) is then produced for each population j by the following formula:
   LR=\tilde{P}ⱼ/\tilde{P}_{GI},
wherein \tilde{P}ⱼ is a joint probability of an individual matching a local population j, adjusted for confidence; and \tilde{P}_{GI} is a joint probability of an individual matching a global index population GI, adjusted for confidence.
Step 5: The MLIs (or LRs) may then be ranked, with the populations having the highest scores considered the best matches for the individuals.

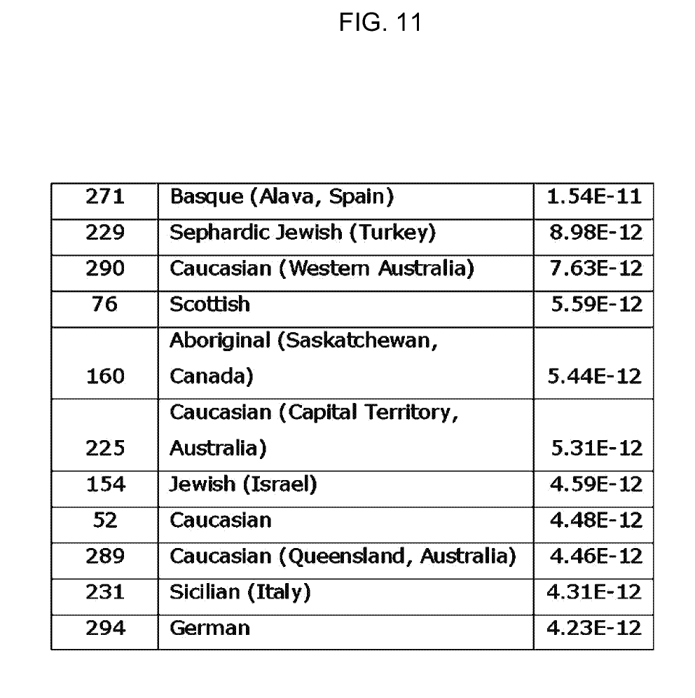

FIG. 11 presents an example of partial matching results for a Basque individual. The numbers to the left of each population are allele values and the numbers to the right of each allele value is its frequency in that particular population sample. The results in FIG. 11 are the ten most likely matching populations, in order with the most likely matching population at the top.

This matching procedure may be repeated multiple times using multiple groups of reference populations. By way of example, a Global Population Match, Native Population Match and/or World Region Match may be performed. For Global Population Match, the individual profile is matched to all populations in the Global Population Database. For Native Population Match, the individual profile is matched to a subset of populations designated as Native (that is, the ones that have experienced minimal post-Colonial admixture in the last 500 years). For World Region Match, the individual profile is matched to four populations identified as representatives of continental clusters.

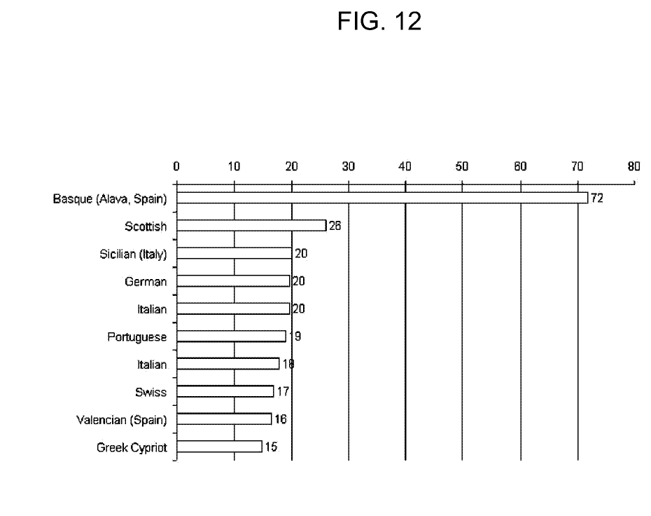

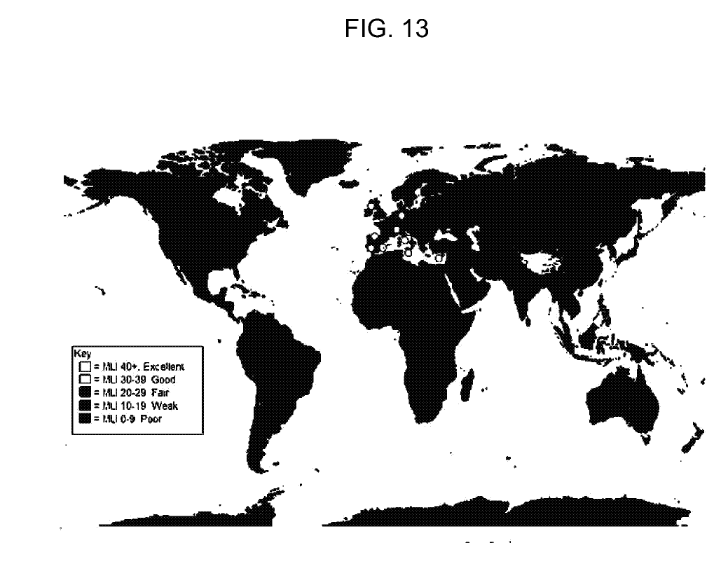

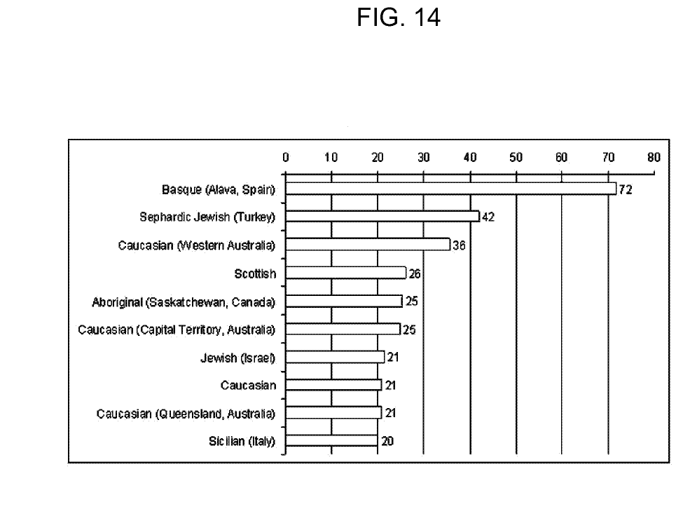

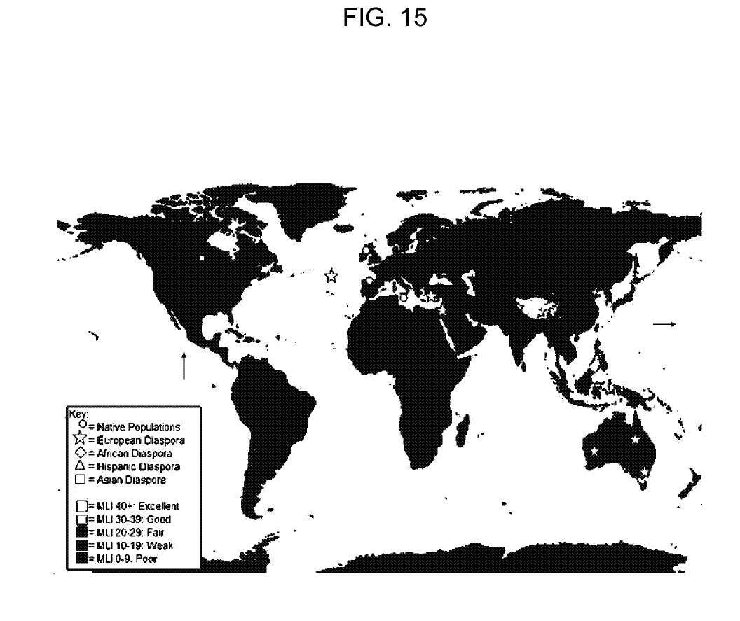

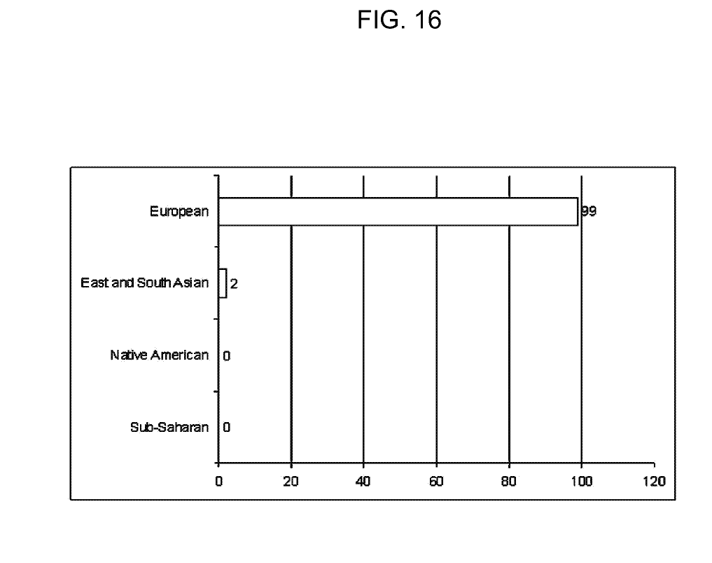

The final output of this analysis for an individual is displayed in FIGS. 12-16. FIGS. 12 and 13 illustrate Native Population Match results. FIGS. 14 and 15 illustrate Global Population Match results. FIG. 16 illustrates World Region Match results.

By using matches presented in multiple formats such as the Global Population Match, Native Population Match, and World Region Match of this example, this technique more accurately identifies the populations where an individual profile is most likely to occur, and estimates an individual's ethnic origin with a high degree of geographical precision. The use of confidence intervals and comparison of each match to a Generic Human Index population allows match results to be measured in terms of likelihood and specificity.

### EXAMPLE 3

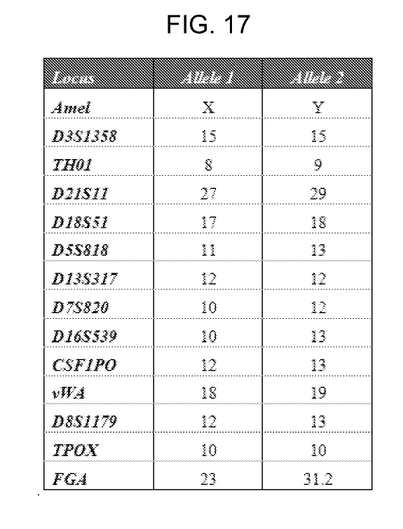

In this example, the DNA of an African individual was used in the present methods. First genetic markers in the individual were determined by sequencing the individual's allele values from a sample of the individual's DNA at 13 autosomal STR loci. Values from nine of these markers were used to compute Native and Global population matches, while values from all thirteen markers were used to compute high resolution World Region matches. The allele values at each locus for the individual are set forth in FIG. 17.

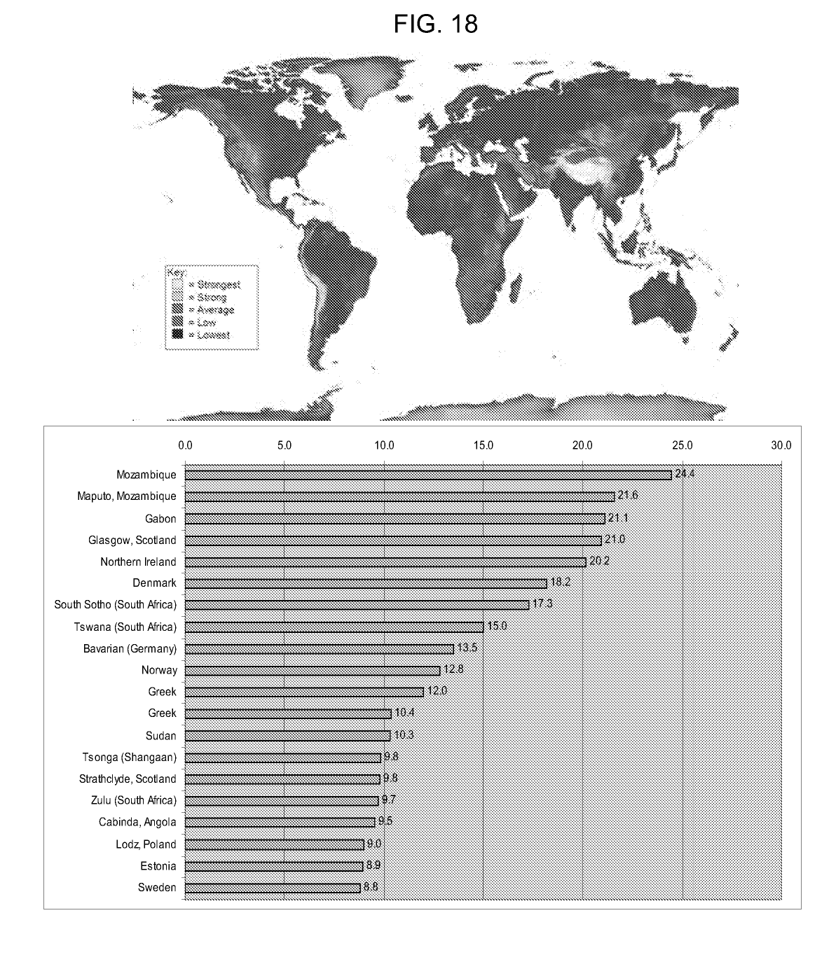

Referring to FIG. 18, the top twenty Native population matches for this individual include both European and African populations. Individual scores do not indicate a person's percentage of individual ethnic groups. Instead, they indicate where a DNA profile is most frequent. As shown in FIG. 18, the strongest match for this individual is with a Mozambique sample, where this individual's DNA profile is 24.4 times as likely as in the world as a whole. However, this DNA profile can be found in other nearby African populations at similar frequencies. For instance, the score for Gabon is 21.1, indicating this DNA profile is 24.4/21.1=1.2 times as likely in Mozambique as in Gabon.

Some nations appear multiple times within these listings. For instance, samples from Mozambique and Maputo, Mozambique appear at similar frequencies. These represent independent population samples. When two samples from the same nation obtain similar scores, this is more evidence of genetic connections to this nation or ethnic group.

For this individual, Mozambique would be the most likely African place of origin, but other ethnic origins such as Gabon, South Sotho, or Sudan cannot be excluded. Because no population is completely isolated from its neighbors, individual DNA profiles often overlap with a number of populations at similar frequencies. These genetic matches provide strong clues as to where this person's ancestors left the strongest genetic traces and where their genetic relatives in Africa live today.

FIG. 18 also shows that this individual's top matches also include European populations, indicating an element of European ancestry. Within Europe, this person's DNA profile is most frequent within Glasgow, Scotland, suggesting Scottish ancestors or ancestors from the British Isles.

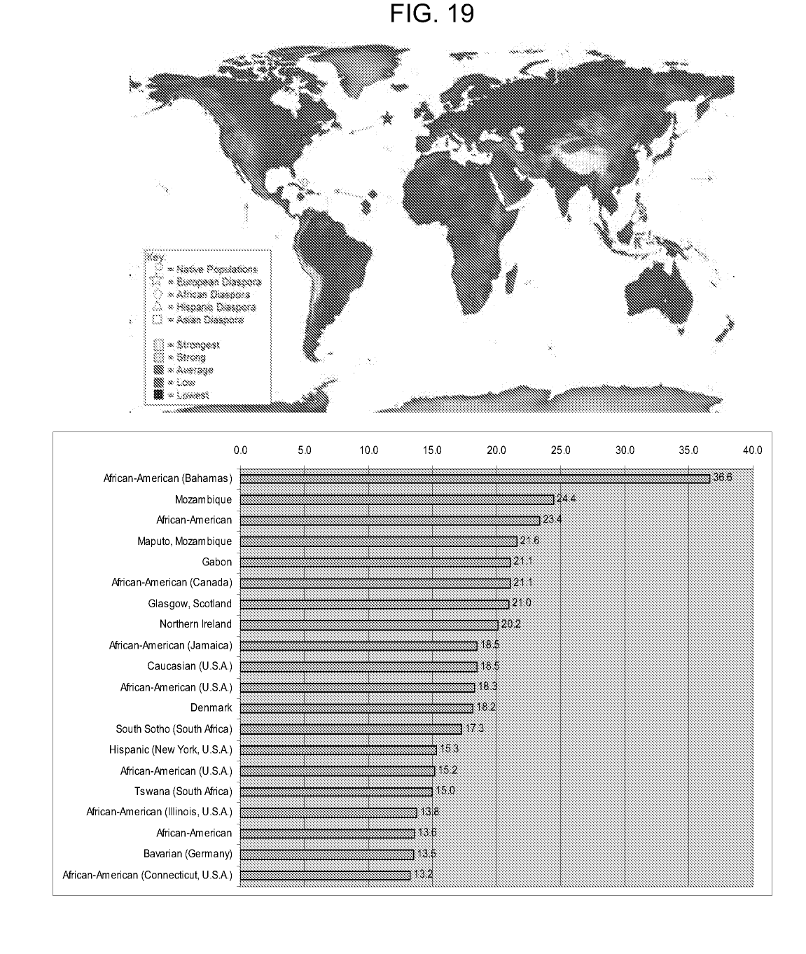

A Global Population Match may then be performed which may provide for example, the individual's top twenty matches in a database of all global populations, including native peoples as well as Diaspora groups that expanded from their homelands and sometimes admixed with other populations in recent history. Results of this individual's Global Population Match are depicted in FIG. 19.

For the individual tested, the Global results include not just native African populations but also the African Diaspora. For instance, this individual's DNA profile can be found at high frequencies in African-Americans living in many places, from the Bahamas to Connecticut. Global Population Matches do not mean this individual's ancestors came from the Bahamas or Connecticut, but indicate places where African-Americans of a similar genetic background live today.

A High Resolution World Region Match was then performed, which measures an individual's genetic connections to for example, twenty-three World Regions. World Regions according to Examples 3 and 4 were defined and determined somewhat differently than in Example 2. In particular, Example 2 identifies World Region “cluster centers,” that is, identifying a population sample that approximates an identified regional group. Examples 3 and 4 define World Regions using medians or means of multiple of member populations rather than a “cluster representative.”

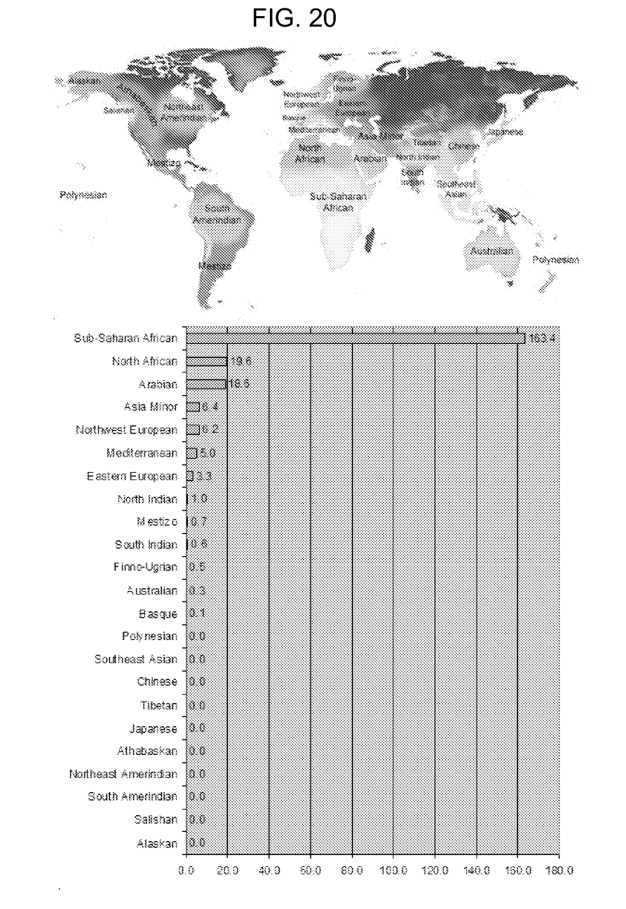

World Region results may provide the best general picture of a person's genetic connections to the world. They can often clarify individual Native and Global population match results when they are difficult to interpret. For instance, this individual's DNA profile (as shown in FIG. 20) is most frequent in Sub-Saharan Africa but can also be found (with scores>1.0) in other regions including Northwest Europe. This is consistent with the distribution of both Native and Global population matches, which are concentrated among populations of African descent but also include British Isles populations. To be more precise, this individual's DNA profile is most frequent in Sub-Saharan, where it is 163.4 times as likely as in the world. Substantial scores (>1.0) also include North Africa, Arabia, Asia Minor, Northwest Europe, the Mediterranean, Eastern Europe and North India. These secondary affiliations indicate this DNA profile can also be found at lower frequencies in other World Regions.

Scores can be compared to each other to give relative frequencies. For instance, this DNA profile is 163.4/19.6=8.3 times as frequent in Sub-Saharan Africa as in North Africa. All scores were measured against the Generic Human Index (GHI or GI) of 1.0. Scores above 1.0 are more frequent in that region than in the world, while scores below 1.0 are more frequent in the world than in that region. For instance, this individual's score for the Basque region is 0.1, indicating this DNA profile is 1.0/0.1=10 times as likely in the world as in the Basque region.

The results for this person provide a detailed and comprehensive picture of their African-American ancestry including their closest genetic relatives amongst ethnic groups in Africa, Europe and the African Diaspora as well as precise measurements of where their DNA profile can be found in 23 World Regions.

### EXAMPLE 4

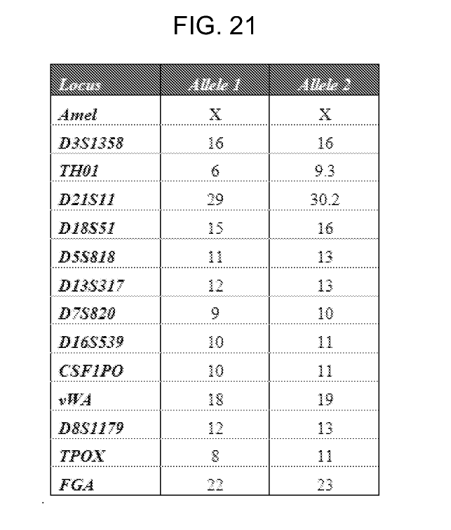

In this example, the DNA of a European individual was used in the present methods. First genetic markers in this individual were determined by sequencing the individual's allele values from a sample of the individual's DNA at 13 autosomal STR loci. The allele values at each locus for the individual are set forth in FIG. 21. For instance, at locus TH01, this individual has inherited one allele of length 6 (6 repeats) and an allele of length 9.3 (9.3 repeats). Values from nine of these markers were used to compute Native and Global population matches, while values from all thirteen markers were used to compute high resolution World Region matches.

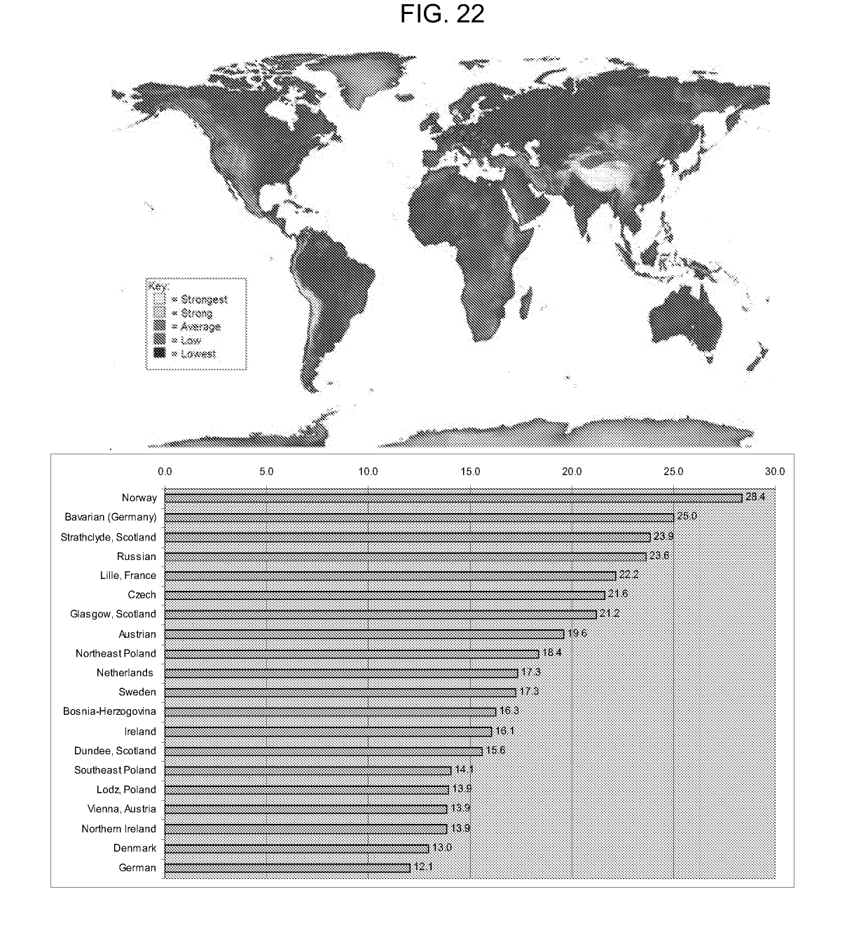

Referring to FIG. 22, this individual's top twenty matches in a database of all native populations that have experienced minimal movement and admixture in the last 500 years were determined by the present methods. Individual matches do not necessarily indicate recent social or cultural affiliation with a particular ethnicity. Rather, the geographical distribution of the individual's Native Population Match results indicates his most likely deep ancestral origins.

The top twenty Native population matches for this individual all fall within Europe. The strongest match is with a Norwegian sample, where this individual's DNA profile is 28.4 times as likely as in the world as a whole. However, this DNA profile can be found in other nearby European nations at similar frequencies. For instance, the score for Sweden is 17.3, indicating this DNA profile is 28.4/17.3=1.64 times as likely in Norway as in Sweden.

For this individual, Norway would be the most likely population of origin, but other ethnic origins such as Austrian, Irish or Dutch cannot be excluded. It is also possible this individual could be of Italian or French heritage but has inherited genetic markers that are more typical of more northerly parts of Europe.

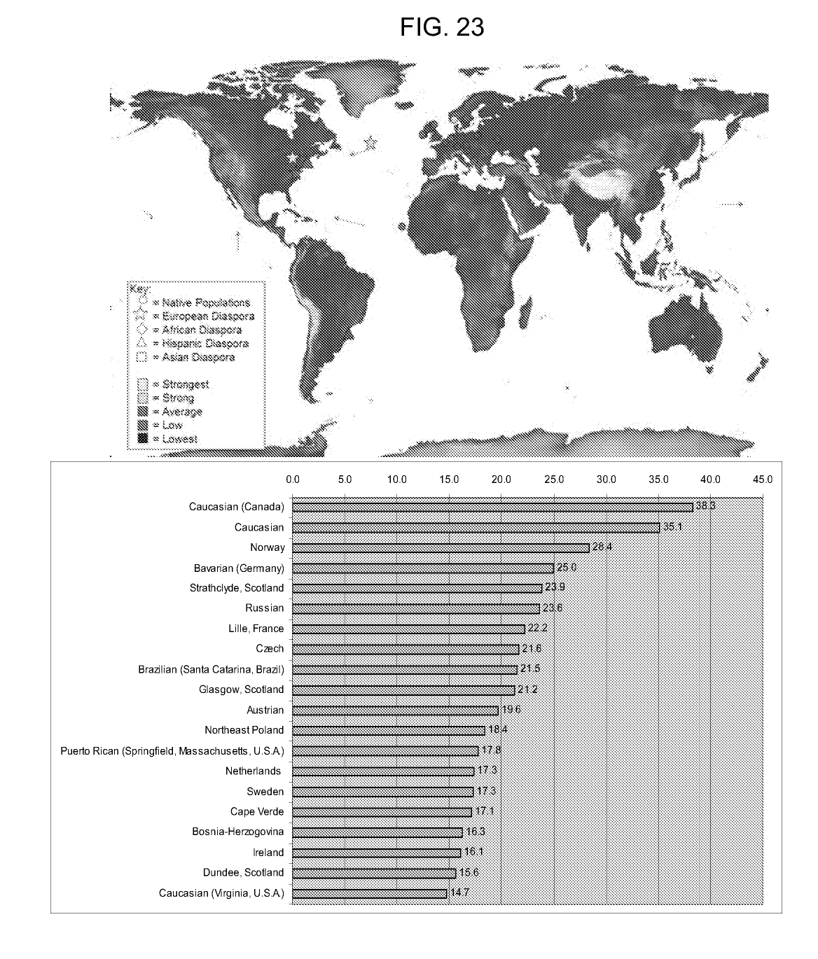

Next, a Global Population Match was performed. The individual's top twenty matches in a database of all global populations, including native peoples as well as Diaspora groups that expanded from their homelands and sometimes admixed with other populations in recent history were provided. These Global results (as depicted in FIG. 23) include not just native European populations but also the European Diaspora. For instance, this individual's DNA profile can be found in Canadian Caucasians, Brazilians from Santa Catarina, Puerto Ricans and Virginia Caucasians at similar frequencies. Global Population Matches do not mean this individual's ancestors came from the Brazil or Virginia, but indicate places where Caucasians of a similar genetic background live today.

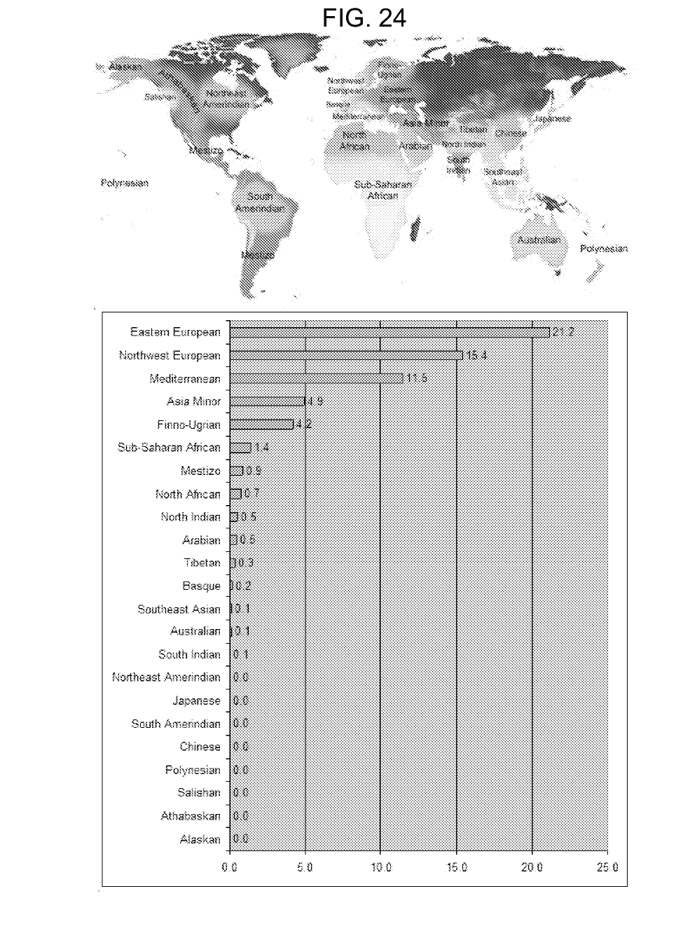

A High Resolution World Region Match was then performed, which measures an individual's genetic connections to for example, twenty-three World Regions. As depicted in FIG. 24, this individual's DNA profile is most frequent in Eastern Europe and Northwest Europe. This is consistent with the distribution of both Native and Global population matches, which are concentrated within these regions. To be more precise, this individual's DNA profile is most frequent in Eastern Europe, where it is 21.2 times as likely as in the world. Substantial scores (>1.0) also include Northern Europe, the Mediterranean, Asia Minor, Finno-Ugrian and Sub-Saharan African. These secondary affiliations indicate this DNA profile can also be found at lower frequencies in other World Regions.

Scores can be compared to each other to give relative frequency. For instance, the DNA profile for this individual is 21.2/15.4=1.37 times as frequent in Eastern Europe as in Northwestern Europe. This indicates that while this person's DNA is most common in Eastern Europe, it is nearly as common in Northwestern Europe. However, this DNA profile is 21.2/4.2=5.0 times as frequent in Eastern Europe as in the Finno-Ugrian region, indicating a stronger difference between these two regions.

All scores were measured against the Generic Human Index of 1.0. Scores above 1.0 are more frequent in that region than in the world, while scores below 1.0 are more frequent in the world than in that region. For instance, this individual's score for North India is 0.5, indicating this DNA profile is 1.0/0.5=2 times as likely in the world as in North India.

The results for this person provide a detailed and comprehensive picture of their European ancestry including their closest genetic relatives amongst ethnic groups in Europe and the European Diaspora as well as precise measurements of where their DNA profile can be found in 23 World Regions.

### EXAMPLE 5

The following is an example of a weak allele that according to certain embodiments would not be used in calculating matches. Let pⱼ denote the proportion of individuals having specific allele value z in population j. The allele “z” is a “weak allele,” and therefore will not be used in the calculations and methods herein, because it fails the following mathematical criteria:
a) pₘₐₓ/p₉₅<3, where pₘₐₓ is the maximum frequency observed in all populations at allele z of locus Z and p₉₅ is the 95% percentile value of the frequencies.
b) at least 90% of the top 20 populations with the highest pⱼ values are in at most two World Regions.

In particular, the following allele value 13 of Gene D3S1358 is a “weak allele” because it fails both criteria as follows: as shown in Table 1, the ratio between the maximum frequency and the 95th percentile is 7.25, which is much larger than 3; as shown in Table 2, the top two World Regions represent only 65% (40% Indian and 25% Mediterranean) of the populations in the top twenty, that is, the twenty populations having the highest frequencies.

TABLE 1
Locus
D3S1358
Allele Value
13
pₘₐₓ
0.0366
p₉₅
0.0051
pₘₐₓ/p₉₅
7.25

TABLE 2

| Population | Frequency | World Region | World Region | # in Top 20 | % of Top 20 |
|---|---|---|---|---|---|
| Katkari Tribal | 0.0366 | Indian | Indian | 8 | 40.00% |
| Uttar Pradesh Khatri | 0.0341 | Indian | Mediterranean | 5 | 25.00% |
| Khandait Orissa | 0.0284 | Indian | African | 3 | 15.00% |
| Oraon Chotanagpur | | | | | |
| Plateau | 0.0245 | Indian | Middle Eastern | 2 | 10.00% |
| Tutsi | 0.0169 | African | Mestizo | 1 | 5.00% |
| African Cape Town | 0.0143 | African | Southeast Asian | 1 | 5.00% |
| Qatar | 0.0114 | Middle Eastern | | | |
| Muslim Karnataka India | 0.0104 | Indian | | | |
| Maheli Tribal Bengal | 0.0103 | Indian | | | |
| Baniya Bihar | 0.0098 | Indian | | | |
| Kuvi Khond Tribal | | | | | |
| Orissa | 0.0096 | Indian | | | |
| Hutu | 0.0092 | African | | | |
| Thai | 0.0077 | Southeast Asian | | | |
| Mestizo Ecuador | 0.0071 | Mestizo | | | |
| Emilia Romagna Italy | 0.0071 | Mediterranean | | | |
| Calabria Italy | 0.0070 | Mediterranean | | | |
| Basque Alava | 0.0051 | Mediterranean | | | |
| Lazio Italy | 0.0051 | Mediterranean | | | |
| Iranian | 0.0050 | Middle Eastern | | | |
| Basque Guipuzcoa | 0.0049 | Mediterranean | | | |

Both criteria may vary. For example, as can be seen by this example, the second criterion is designed to ensure that an allele value is strongly associated with a small number of populations. Although the number of populations considered may be more or fewer than twenty, and the percentages required for the criteria to be met may vary, the goal is to make sure an allele value is strongly associated with only a small number of populations versus being spread all over the world.

Although the invention has been described in example embodiments, many additional modifications and variations would be apparent to those skilled in the art. For example, modifications may be made for example to the methods described herein including the addition of or changing the order of various steps. Modifications may be made to the example statistical analyses provided herein. Other examples of possible modifications may include modifications to the output that demonstrates calculated results, such as rankings, lists, maps, etc. It is therefore to be understood that this invention may be practiced other than as specifically described. Thus, the present embodiments should be considered in all respects as illustrative and not restrictive.

What is claimed is:

1. A method of determining an individual's relative likelihood of a genetic match with one or more local populations as compared to a generic index population comprising:
determining a genetic likelihood of the individual belonging to at least one local population;
determining a genetic likelihood of the individual belonging to a generic index population; and
comparing the likelihood of the individual belonging to the at least one local population to the likelihood of the individual belonging to the generic index population to determine the individual's relative likelihood of a genetic match with the one or more local populations.

2. The method of claim 1, wherein the genetic likelihood of the individual belonging to at least one local population, is determined by comparing genetic markers present in the individual at a plurality of genetic loci, to the frequency of such genetic markers occurring in the at least one local population.

3. The method of claim 2, wherein the genetic likelihood of the individual belonging to a generic index population, is determined by comparing genetic markers present in the individual at a plurality of genetic loci, to the frequency of such genetic markers occurring in the generic index population.

4. The method of claim 1, wherein comparing the likelihood of the individual belonging to the at least one local population to the likelihood of the individual belonging to a generic index population comprises
dividing the likelihood of the individual belonging to a first local population by the likelihood of the individual belonging to a generic index population to determine a relative likelihood ratio of the individual belonging to the first local population.

5. The method of claim 4, further comprising
comparing the likelihood of the individual belonging to a second or more local population to the likelihood of the individual belonging to a generic index population to determine relative likelihood ratios of the individual belonging to each of the second or more local populations; and
ranking the relative likelihood ratios of the individual belonging each local population.

6. The method of claim 4, wherein a relative likelihood ratio LR of an individual belonging to a local population as compared to a generic population is calculated using the following formula:
LR=\tilde{P}ⱼ/\tilde{P}_{GI},
wherein \tilde{P}ⱼ is a joint probability of an individual matching a local population j, adjusted for confidence; and \tilde{P}_{GI} is a joint probability of an individual matching a global index population, adjusted for confidence.

7. The method of claim 1, wherein the genetic likelihood of the individual belonging to at least one local population is determined by a method comprising:
extracting from a database, frequencies p matching the individual's allele value at each locus w, w=1 . . . 2N, where N is a number of genetic loci for which data is collected from the individual, for each local population; and
determining a joint probability Pⱼ of an individual matching a local population j by multiplying the extracted frequencies p_{w\j} using the following formula
$${P}_{j} = {\prod }_{w = 1}^{2 N} {p}_{w \vert j} .$$

8. The method of claim 7, further comprising adjusting the joint probability Pj for confidence.

9. The method of claim 8, wherein the joint probability Pj of an individual matching a local population j, is adjusted by determining a lower bound of a confidence interval to arrive at a joint matching probability \tilde{P}ⱼ, wherein the joint matching probability \tilde{P}ⱼ is determined by a method using the following formula:
$${\tilde{P}}_{j} = exp \{ log {P}_{j} - {Z}_{C} \sqrt{\frac{1}{{n}_{j}} {\sum }_{w = 1}^{2 N} \frac{1 - {p}_{w \vert j}}{{p}_{w \vert j}}} \}$$
wherein p_{w\j} is a frequency of the individual's allele value at each locus w in population j, w=1 . . . 2N, where N is the number of genetic loci for which data are collected from the individual, nⱼ is the number of individuals in population j for which genetic data were collected, and Z_{C} is a z-score corresponding to the C confidence level.

10. The method of claim 1, wherein the genetic likelihood of the individual belonging to a generic index population is determined by a method comprising:
extracting from a database, frequencies p matching the individual's allele value at each locus w, w=1 . . . 2N, where N is a number of genetic loci for which data is collected from the individual, for the generic index population GI; and
determining a joint probability P_{GI} of an individual matching the generic index population by multiplying the extracted frequencies p_{w\GI} using the following formula
$${P}_{GI} = {\prod }_{w = 1}^{2 N} {p}_{w \vert GI} .$$

11. The method of claim 10, further comprising adjusting the joint probability P_{GI} for confidence.

12. The method of claim 11, wherein the joint probability of an individual matching a global population \tilde{P}_{GI}, as adjusted by determining the lower bound of a confidence interval, is determined by a method using the following formula:
$${\tilde{P}}_{GI} = exp \{ log {P}_{GI} - {Z}_{C} \sqrt{\frac{1}{{n}_{GI}} {\sum }_{w = 1}^{2 N} \frac{1 - {p}_{w \vert GI}}{{p}_{w \vert GI}}} \}$$
wherein P_{GI} is the joint probability of an individual matching the generic index population, p_{w\GI} is a frequency of matching the individual's allele value at each locus w, w=1 . . . 2N, N is the number of genetic loci for which data is collected from the individual, and n_{GI} is determined by the following formula:
$${n}_{GI} = \frac{1}{K} {\sum }_{j = 1}^{K} {n}_{j}$$
where K is a number of local populations used to calculate the generic index population, and nⱼ is a number of individuals comprising local population j.

13. The method of claim 3, wherein the genetic markers in the individual are determined by sequencing the individual's allele values from a sample of the individual's DNA at N autosomal loci, wherein N is any positive integer.

14. The method of claim 3, wherein the genetic markers in the individual are determined by sequencing the individual's allele values from a sample of the individual's DNA at N autosomal STR loci, wherein N is any positive integer.

15. The method of claim 3, wherein the genetic markers in the individual are determined by sequencing the individual's allele values from a sample of the individual's DNA at N autosomal SNP loci, wherein N is any positive integer.

16. The method of claim 1, wherein the local population is defined by a method comprising using a multivariate clustering algorithm by separating a local population database into K groups.

17. The method of claim 1, wherein the generic index population is calculated as an average or median of all local populations in a database.

18. The method of claim 1, wherein a first likelihood is determined of the individual belonging to a first local population and a second likelihood is determined of the individual belonging to a second local population, further comprising
comparing the first likelihood to the second likelihood to determine a relative likelihood of the individual belonging to the first local population as compared to the second local population.

19. The method of claim 1, wherein the local population is a world region population and the generic index population is an average or median of all world region populations.

20. The method of claim 3, wherein the frequency of genetic markers occurring in the generic index population is determined by a method comprising determining frequencies of alleles occurring at each of N loci for multiple local populations and averaging or determining the median of frequencies for each allele for all of the multiple local populations.

21. The method of claim 1, wherein each local population of is a breed of organisms, and the generic index population is a species of organisms.

22. The method of claim 21, wherein the individual is an individual dog, each local population is a breed of dogs, and the generic index population is dogs.

23. The method of claim 1, wherein the generic index population is selected from the group consisting of a kingdom, phylum, class, order, family, genus, species, and any subdivisions thereof.

24. An apparatus comprising a server comprising software capable of
determining a first likelihood of an individual belonging to a local population by comparing genetic markers present in the individual to a frequency of such genetic markers occurring in the local population;
determining a second likelihood of the individual belonging to a generic index population by comparing the genetic markers present in the individual to a frequency of such genetic markers occurring in the generic index population; and
comparing the first likelihood to the second likelihood.

25. A system comprising
a server coupled to a database;
wherein said database includes information regarding genetic markers occurring in at least one local population and information regarding genetic markers occurring in a generic index population; and
wherein the server comprises software capable of
determining a first likelihood of an individual belonging to a local population by comparing genetic markers present in the individual to a frequency of such genetic markers occurring in the local population; and
determining a second likelihood of the individual belonging to a generic index population by comparing the genetic markers present in the individual to a frequency of such genetic markers occurring in the generic index population.

26. The system of claim 25, wherein the software is further capable of comparing the first likelihood to the second likelihood.

27. The system of claim 25, wherein the information regarding genetic markers occurring in a generic index population is derived from information regarding genetic markers occurring in at least one local population.

28. A machine-readable medium comprising code segments embodied on a medium that, when read by a machine, cause the machine to perform the method of claim 1.

29. The machine-readable medium of claim 28, wherein the medium is a computer readable medium and the code segments comprise a program for performing the method of claim 1.

30. A kit comprising:
at least one device for determining genetic markers of an individual; and
a machine readable medium comprising
a medium and
a program capable of comparing genetic markers of the individual to at least one local population and comparing the genetic markers of the individual to a generic index population, to determine a relative genetic likelihood of the individual belonging to the at least one local population as compared to the generic index population.

31. The kit of claim 30, wherein the device for determining genetic markers of an individual comprises at least one device selected from the group consisting of a sample collector and a device for reading DNA from a sample collector.

32. The kit of claim 31, wherein the sample collector is a swab capable of collecting DNA of an individual.
